(19) United States
(12) Patent Application Publication
(10) Pub. No.: US 2009/0123920 A1
(43) Pub. Date: May 14, 2009

(54) JAK2 MUTATIONS

(76) Inventor: Maher Albitar, Coto De Caza, CA (US)

Correspondence Address: FOLEY & LARDNER LLP, P.O. BOX 80278, SAN DIEGO, CA, 92138-0278, US

(21) Appl. No.: 11/937,400

(22) Filed: Nov. 8, 2007

(51) Int. Cl. C12Q 1/68; C07H 21/04; C07K 7/06; C07K 7/08; C07K 14/435

(52) U.S. Cl. 435/6; 536/23.1; 530/328; 530/327; 530/326; 530/325; 530/324; 530/350

(57) ABSTRACT

The invention disclosed herein is based on the identification of novel mutations in the JAK2 gene and JAK2 protein. The invention provides methods and compositions useful for diagnosing neoplastic diseases including, for example, myeloproliferative diseases. The invention also provides methods and compositions useful for determining a prognosis of an individual diagnosed as having a neoplastic disease.

### FIELD OF INVENTION

This invention relates to the field of cancer diagnosis and treatment.

### BACKGROUND OF INVENTION

The following discussion of the background of the invention is merely provided to aid the reader in understanding the invention and is not admitted to describe or constitute prior art to the present invention.

Certain neoplastic diseases including non-CML myeloproliferative diseases (MPDs) such as polycythemia vera (PV), essential thrombocythemia (ET), and chronic idiopathic myelofibrosis (IMF) and as of yet unclassified myeloproliferative diseases (MPD-NC) are characterized by an aberrant increase in blood cells. See e.g., Vainchenker and Constantinescu, Hematology (American Society of Hematology), 195-200 (2005). This increase is generally initiated by a spontaneous mutation in a multipotent hematopoetic stem cell located in the bone marrow. Id. Due to the mutation, the stem cell produces far more blood cells of a particular lineage than normal, resulting in the overproduction of cells such as erythroid cells, megakaryocytes, granulocytes and monocytes. Some symptoms common to patients with MPD include enlarged spleen, enlarged liver, elevated white, red and/or platelet cell count, blood clots (thrombosis), weakness, dizziness and headache. Diseases such as PV, ET and IMF may presage leukemia, however the rate of transformation (e.g., to blast crisis) differs with each disease. Id.

The specific gene and concomitant mutation or mutations responsible for many MPDs is not known. However, a mutation in the Janus kinase 2 (JAK2) gene, a cytoplasmic, nonreceptor tyrosine kinase, has been identified in a number of MPDs. For example, this mutation has been reported in up to 97% of patients with PV, and in greater than 40% of patients with either ET or IMF. See e.g., Baxter et al., Lancet 365:1054-1060 (2005); James et al., Nature 438:1144-1148 (2005); Zhao, et al., J. Biol. Chem. 280(24):22788-22792 (2005); Levine et al., Cancer Cell, 7:387-397 (2005); Kralovics, et al New Eng. J. Med. 352(17):1779-1790 (2005); Jones, et al., Blood 106:2162-2168 (2005); Steensma, et al., Blood 106:1207-2109 (2005).

The Janus kinases are a family of tyrosine kinases that play a role in cytokine signaling. For example. JAK2 kinase acts as an intermediary between membrane-bound cytokine receptors such as the erythropoietin receptor (EpoR), and down-stream members of the signal transduction pathway such as STAT5 (Signal Transducers and Activators of Transcription protein 5). Slee, e.g. Schindler, C. W., J. Clin Invest. 109:1133-1137 (2002); Tefferi and Gilliland, Mayo Clin. Proc. 80:947-958 (2005); Giordanetto and Kroemer, Protein Engineering, 15(9):727-737 (2002). JAK2 is activated when cytokine receptor/ligand complexes phosphorylate the associated JAK2 kinase. Id. JAK2 can then phosphorylate and activate its substrate molecule, for example STAT5, which enters the nucleus and interacts with other regulatory proteins to affect transcription. Id.; Nelson, M. E., and Steensma, D. P., Leuk. Lymphoma 47:177-194 (2006).

In one JAK2 mutant, a valine (codon “GTC”) is replaced by a phenylalanine (codon “TTC”) at amino acid position 617 (the “V617F mutant”). Baxter et at., Lancet 365:1054-1060 (2005). Amino acid 617 is located in exon 12 which includes a pseudokinase, auto-inhibitory (or negative regulatory) domain termed JH2 (Jak Homology 2 domain). Id.; James et al., Nature 438:1144-1148 (2005). Though this domain has no kinase activity, it interacts with the JH1 (Jak Homology 1) domain, which does have kinase activity. Baxter et al., Lancet 365:1054-1060 (2005). Appropriate contact between the two domains in the wild-type protein allows proper kinase activity and regulation; however, the V617F mutation causes improper contact between the two domains, resulting in constitutive kinase activity in the mutant JAK2 protein. Id.

A variety of different approaches and a large body of evidence suggest that, when present, the JAK2 V617F mutation contributes to the pathogenesis of MPD. See e.g., Kaushansky, Hematology (Am Soc Hematol Educ Program), 533-7 (2005). The mutation has been detected from blood samples, bone marrow and buccal samples (see, e.g, Baxter et al., Lancet 365:1054-1060 (2005); James et al., Nature 438:1144-1148 (2005); Zhao et al., J. Biol. Chem. 280(24):22788-22792 (2005); Levine et al., Cancer Cell, 7:387-397 (2005): Kralovics, et al., New Eng. J. Med. 352(17):1779-1790 (2005)), and homozygous and heterozygous cell populations have been reported in MPD patients. Baxter et al., Lancet 365:1054-1060 (2005).

### SUMMARY OF THE INVENTION

The present invention is based on the discovery of previously unknown mutations in the JAK2 gene and protein. Specifically, the JAK2 gene mutations include the G1920T/C1922T double mutation, the G1920A mutation, and the T1923C mutation which result in the V617F, V617I, and C618R amino acid substitutions in the JH2 domain of the JAK2 protein, respectively. The invention further provides methods and compositions useful in the diagnosis and prognosis of neoplastic diseases including, for example, myeloproliferative diseases.

Accordingly, in one aspect, the invention provides a method for diagnosing a neoplastic disease comprising determining the presence or absence of one or more mutations in the JAK2 nucleic acid of a patient, said mutation selected from the group consisting of G1920A, T1923C, and G1920T/C1922T.

In another aspect, the invention provides a method for determining a prognosis of an individual diagnosed with a neoplastic disease comprising determining the presence or absence of one or more mutations in the JAK2 nucleic acid of a patient, said mutation selected from the group consisting of G1920A, T1923C, and G1920T/C1922T, and using the mutation status to predict the clinical outcome for the individual.

In some embodiments, the JAK2 nucleic acid comprises the T1923C mutation in combination with the G1920T mutation, the G1920T/C1922T mutation, or the G1920A mutation.

In preferred methods for prognosis, the JAK2 mutation status is combined with at least one other clinical parameter. Suitable clinical parameters include, for example, age and percent blast cell count.

In another aspect, the invention provides a method for diagnosing a neoplastic disease comprising determining the presence or absence of a mutation in the JAK2 protein of a patient, said mutation selected from the group consisting of V617A and C618R.

In some embodiments, the JAK2 protein contains the C618R mutation in addition to another JAK2 mutation including, for example, the V617A mutation and the V617F mutation.

In other embodiments, the one or more mutations in either the JAK2 nucleic acid and/or the JAK2 protein affect JAK2 kinase activity. Preferably the JAK2 kinase activity is reduced. More preferably, the JAK2 kinase activity is reduced because of reduced interaction between the JH2 (pseudokinase) domain and the JH1 domain of JAK2.

The neoplastic diseases which are the subject of the inventive methods include myeloproliferative diseases including, for example, polycythemia vera, essential thrombocythemia, idiopathic myelofibrosis, and unclassified myeloproliferative disease.

In other embodiments, the JAK2 protein and/or nucleic acid is obtained from a biological sample (e.g., a body fluid) from the patient. In other embodiments, the body fluid is an acellular body fluid.

In another aspect, the invention provides an isolated JAK2 nucleic acid. Preferred JAK2 nucleic acids comprise at least 12 contiguous nucleotides of SEQ ID NO: 1, wherein the nucleic acid comprises a mutation selected from the group consisting of G1920A, T1923C, and G1920T/C1922T, or a complement thereof. Preferably, the JAK2 nucleic acid comprises the T1923C mutation in combination with the G1920T mutation, the G1920T/C 1922T mutation, or the G1920A mutation. In other preferred embodiments, the JAK2 nucleic acid comprises at least 14, 16, 18, 20, 22, 25, 30 40, 50, 75, 100, 150, 200, 250, 500, or more nucleotides. Optionally, the JAK2 nucleic acid may further contain a detectable label (e.g., a fluorescent label).

In another aspect, the invention provides an isolated JAK2 polypeptide. Preferred JAK2 polypeptides comprise at least 10 contiguous amino acids of SEQ ID NO: 3, wherein the polypeptide comprises the V617I mutation and/or the C618R mutation. In other preferred embodiments, the JAK2 polypeptide comprises at least 12, 14, 16, 18, 20, 22, 25, 30 40, 50, 75, 100, 150, 200, 250, 500, or more amino acids.

As used herein, “plasma” refers to acellular fluid found in blood. “Plasma” may be obtained from blood by removing whole cellular material from blood by methods known in the art (e.g., centrifugation, filtration, and the like). As used herein, “peripheral blood plasma” refers to plasma obtained from peripheral blood samples.

As used herein, “serum” includes the fraction of plasma obtained after plasma or blood is permitted to clot and the clotted faction is removed.

The term “nucleic acid” or “nucleic acid sequence” refers to an oligonueleotide, nucleotide or polynucleotide, and fragments or portions thereof, which may be single or double stranded, and represent the sense or antisense strand. A nucleic acid may include DNA or RNA, and may be of natural or synthetic origin. For example, a nucleic acid may include mRNA or cDNA. Nucleic acid may include nucleic acid that has been amplified (e.g., using polymerase chain reaction). The convention “Twt###NTmut” is used to indicate a mutation that results in the wild-type nucleotide NTwt at position ### in the nucleic acid being replaced with mutant NTmut. For example, G1920A refers to a mutation at nucleotide position 1920 in SEQ ID NO: 1 (the reference nucleotide sequence) in which the wild-type guanine is changed (mutated) to an adenine.

An “amino acid sequence” refers to a polypeptide or protein sequence. The convention “AAwt###AAmut” is used to indicate a mutation that results in the wild-type amino acid AAwt at position ### in the polypeptide being replaced with mutant AAmut. For example, C618R refers to a mutation at amino acid position 618 of SEQ ID NO: 3 (the reference polypeptide sequence) in which the wild-type cysteine is changed (mutated) to an arginine.

The term “wild-type” refers to a gene or a gene product that is most frequently observed in a population and not associated with disease. “Wild-type” may also refer to the sequence at a specific nucleotide position or positions, or the sequence at a particular codon position or positions, or the sequence at a particular amino acid position or positions. For example, a gene can be “wild-type” at nucleotide position 1849 or at codon 617. As used herein, “mutant,” “modified” or “polymorphic” refers to a gene or gene product which displays modifications in sequence and or functional properties (i.e., altered characteristics) when compared to the wild-type gene or gene product. “Mutant,” “modified” or “polymorphic” also refers to the sequence at a specific nucleotide position or positions, or the sequence at a particular codon position or positions, or the sequence at a particular amino acid position or positions.

A “mutation” is meant to encompass at least a single nucleotide variation in a nucleic acid sequence relative to the normal sequence or wild-type sequence. A mutation may include a substitution, a deletion, an inversion or an insertion. With respect to an encoded polypeptide, a mutation may be “silent” and result in no change in the encoded polypeptide sequence or a mutation may result in a change in the encoded polypeptide sequence. For example, a mutation may result in a substitution in the encoded polypeptide sequence. A mutation may result in a frameshift with respect to the encoded polypeptide sequence.

As used herein the term “codon” refers to a sequence of three adjacent nucleotides (either RNA or DNA) constituting the genetic code that determines the insertion of a specific amino acid in a polypeptide chain during protein synthesis or the signal to stop protein synthesis. The term “codon” is also used to refer to the corresponding (and complementary) sequences of three nucleotides in the messenger RNA into which the original DNA is transcribed.

The term “substantially all” means between about 60-100%, more preferably, between about 70-100%; more preferably between about 80-100%, more preferably between about 90-100%, and more preferably between about 95-100%.

An oligonucleotidc (e.g., a probe or a primer) that is specific for a target nucleic acid will “hybridize” to the target nucleic acid under suitable conditions. As used herein, “hybridization” or “hybridizing” refers to the process by which a oligonucleotide single strand anneals with a complementary strand through base pairing under defined hybridization conditions.

By “isolated”, when referring to a nucleic acid (e.g., an oligonucleotide) is meant a nucleic acid that is apart from a substantial portion of the genome in which it naturally occurs. For example, any nucleic acid that has been produced synthetically (e.g., by serial base condensation) is considered to be isolated. Likewise, nucleic acids that are recombinantly expressed, produced by a primer extension reaction (e.g., PCR), or otherwise excised from a genome are also considered to be isolated.

“Specific hybridization” is an indication that two nucleic acid sequences share a high degree of complementarity. Specific hybridization complexes form under permissive annealing conditions and remain hybridized after any subsequent washing steps. Pennissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tm) for the specific sequence at a defined ionic strength and pH. The Tm is the temperature (under defined ionic strength and pH) at which 50% of the target sequence hybridizes to a perfectly matched probe. Equations for calculating Tm and conditions for nucleic acid hybridization are known in the art.

Oligonucleotides used as primers or probes for specifically amplifying (i.e., amplifying a particular target nucleic acid sequence) or specifically detecting (i.e., detecting a particular target nucleic acid sequence) a target nucleic acid generally are capable of specifically hybridizing to the target nucleic acid.

For the JAK2 nucleic acid sequence, a “mutation” means a JAK2 nucleic acid sequence that includes at least one nucleic acid variation as compared to reference sequence GenBank accession number NM004972. For convenience, the cDNA sequence of JAK2 is provided in FIG. 1 (SEQ ID NO: 1), and the pseudokinase domain is provided in FIG. 2 (SEQ ID NO:2). A mutation may include a substitution, a deletion or an insertion. A mutation in JAK2 nucleic acid may result in a change in the encoded polypeptide sequence or the mutation may be silent with respect to the encoded polypeptide sequence. An example of a JAK2 mutation that results in a change in polypeptide sequence includes, but is not limited to V617F. A change in an amino acid sequence may be determined as compared to SEQ ID NO: 3, FIG. 3 as a reference amino acid sequence.

“Determining the presence or absence of one or more mutations” in a nucleic acid also includes detecting the nucleic acid. For example, in determining the presence or absence of a mutation in JAK2, the JAK2 nucleic acid is also detected. Methods of determining the presence or absence of one or more mutations may include a variety of methods known in the art including one or more of reverse transcribing JAK2 RNA to cDNA, amplifying JAK2 nucleic acid, hybridizing a probe or a primer to JAK2 nucleic acid, and sequencing JAK2 nucleic acid.

The term “oligonucleotide” is understood to be a molecule that has a sequence of bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group in this position. Oligonucleotides also may include derivatives, in which the hydrogen of the hydroxyl group is replaced with organic groups, e.g., an allyl group. Oligonucleotides of the method which function as primers or probes are generally at least about 10-15 nucleotides long and more preferably at least about 15 to 25 nucleotides long, although shorter or longer oligonucleotides may be used in the method. The exact size will depend on many factors, which in turn depend on the ultimate function or use of the oligonucleotide. The oligonucleotide may be generated in any manner, including chemical synthesis, DNA replication, reverse transcription, PCR, or a combination thereof. The oligonucleotide may be modified. For example, the oligonucleotide may be labeled with an agent that produces a detectable signal (e.g., a fluorophore).

“Primer” refers to an oligonucleotide that is capable of acting as a point of initiation of synthesis when placed under conditions in which primer extension is initiated (e.g., primer extension associated with an application such as PCR). An oligonucleotide “primer” may occur naturally, as in a purified restriction digest or may be produced synthetically.

A “probe” refers to an oligonucleotide that interacts with a target nucleic acid via hybridization. A probe may be fully complementary to a target nucleic acid sequence or partially complementary. The level of complementarity will depend on many factors based, in general, on the function of the probe. A probe or probes can be used, for example to detect the presence or absence of a mutation in a nucleic acid sequence by virtue of the sequence characteristics of the target. Probes can be labeled or unlabeled, or modified in any of a number of ways well known in the art. A probe may specifically hybridize to a target nucleic acid.

A “target nucleic acid” refers to a nucleic acid molecule containing a sequence that has at least partial complementarity with a probe oligonucleotide and/or a primer oligonucleotide. A probe may specifically hybridize to a target nucleic acid.

As used herein, the term “activation domain” in reference to JAK2 refers generally to a domain involved in cell activation. An example of an activation domain is a kinase or pseudokinase domain.

As used herein, the term “pseudokinase domain” refers to a portion of a polypeptide or nucleic acid that encodes a portion of the polypeptide, where the portion shows homology to a functional kinase but possesses no catalytic activity. A pseudokinase domain may also be referred to as a “kinase-like domain.” An example of a pseudokinase domain is the JAK2 psuedokinase domain, also termed the JH2 domain, represented within SEQ ID NO: 2, FIG. 2.

The term “kinase domain” refers to a portion of a polypeptide or nucleic acid that encodes a portion of the polypeptide, where the portion is required for kinase activity of the polypeptide (e.g., tyrosine kinase activity).

In some methods of the invention, mutations may “affect JAK2 kinase activity.” The affected JAK2 kinase activity may include kinase activity that increases, decreases, becomes constitutive, stops completely or affects greater, fewer or different targets. A mutation that affects kinase activity may be present in a kinase domain or in a domain associated with a kinase domain such as the JAK2 pseudokinase domain.

As used herein the terms “diagnose” or “diagnosis” or “diagnosing” refer to distinguishing or identifying a disease, syndrome or condition or distinguishing or identifying a person having a particular disease, syndrome or condition.

### BRIEF DESCRIPTION OF THE FIGURES

FIG. 1 is a nucleic acid sequence of JAK2.

FIG. 2 is a nucleic acid sequence of the JAK2 pseudokinase domain region.

FIG. 3 is an amino acid sequence of JAK2.

FIG. 4 is an amino acid sequence of the JAK2 pseudokinase domain region.

FIG. 5 is a series of electropherogram tracings. FIG. 5A shows an electropherogram tracing from a wild-type JAK2 gene in which the codon encoding amino acid 617 is GTC (valine). FIG. 5B is an electropherogram tracing of the JAK2 gene from a blood cell sample of an individual indicating heterozygosity at the nucleotide position corresponding to position 1920 of SEQ ID NO: 1. The dot indicates the peak associated with thiamine. FIG. 5C is an electropherogram tracing of a plasma sample from the same individual as used in FIG. 5B, indicating that the individual is homozygous or hemizygous for the thiamine point mutation at position 1920.

FIG. 6 is a series of electropherogram tracings. FIG. 6A is an electropherogram from a wild-type JAK2 gene. FIG. 6B is an electropherogram from an individual having a double point mutation (grey arrows) in codon 617 and a single point mutation (black arrow) in codon 618. The mutated codon 617 and 618 are TTT and CGT, respectively. FIG. 6C is the mutation electropherogram. In each case, the nucleotide numbering refers to Genbank accession NM004972. For reference, position 2343 corresponds to position 1920 of SEQ ID NO: 1.

FIG. 7 is a series of electropherogram tracings. FIG. 6A is an electropherogram from an individual having a G>A point mutation at the nucleotide position corresponding to position 1920 of SEQ ID NO: 1. FIG. 6B is an electropherogram from an individual having a G>T point mutation at the nucleotide position corresponding to position 1920 of SEQ ID NO: 1. FIG. 6C is and electropherogram from a wild-type JAK2 gene. In each case, the nucleotide numbering refers to Genbank accession NM004972. For reference, position 2343 corresponds to position 1920 of SEQ ID NO: 1.

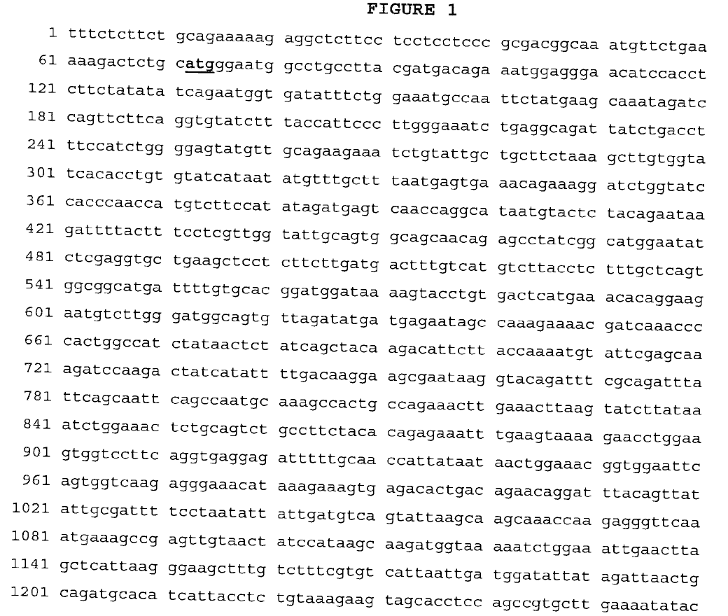

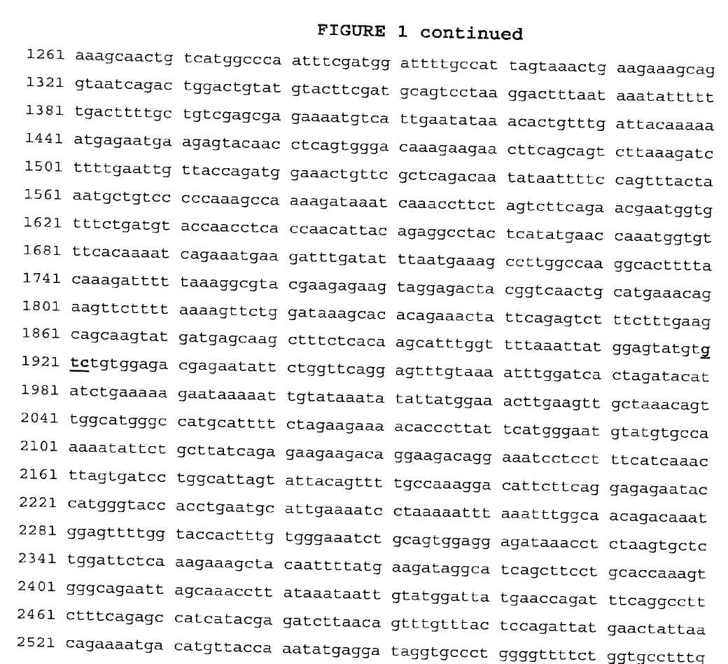

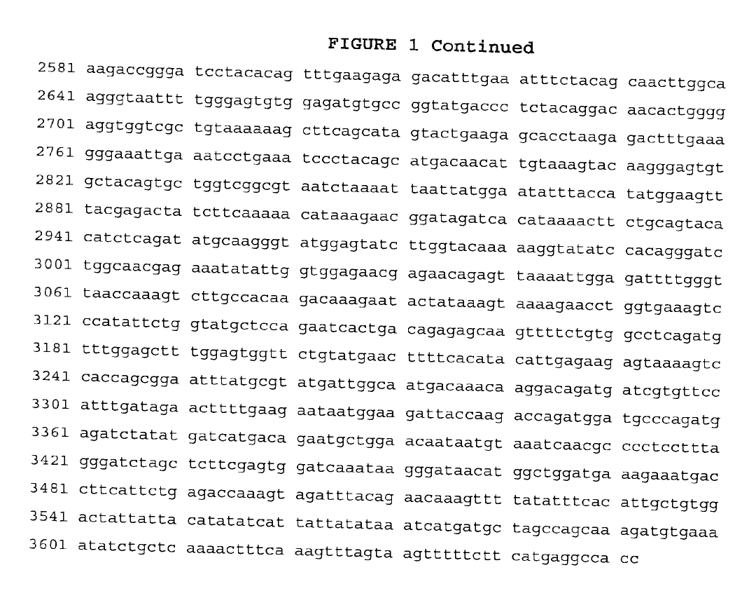

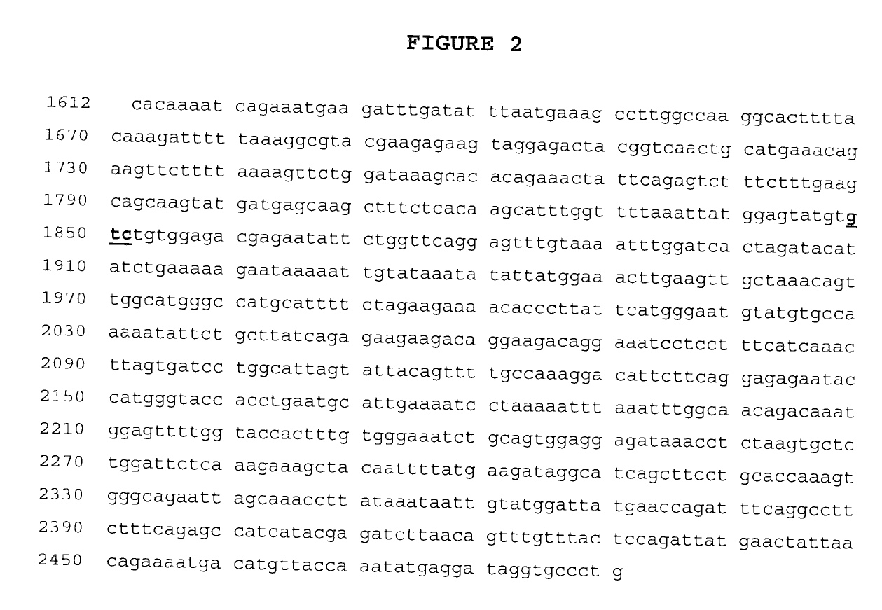

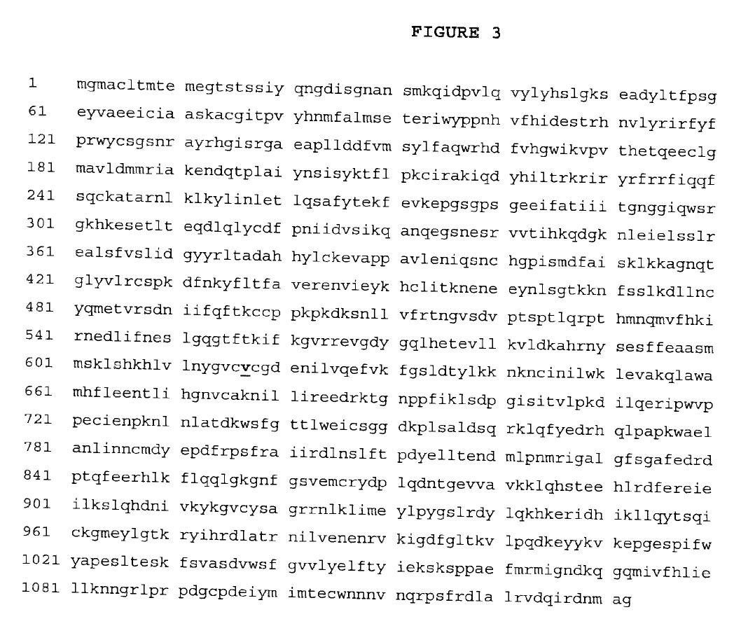

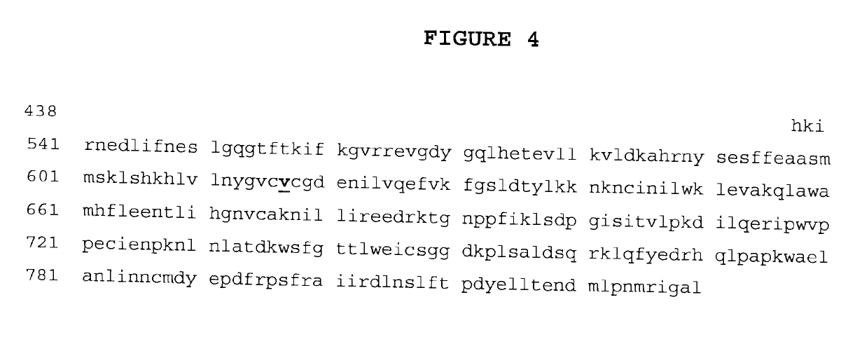

### DETAILED DESCRIPTION OF INVENTION

The present invention is based on the discovery of previously unknown mutations in the JAK2 gene and protein which have been associated with myeloproliferative diseases. Specifically, the JAK2 gene mutations include the G1920T/C1922T double mutation, the G1920A mutation, and the T1923C mutation which result in the V617F, V617I, and C618R amino acid substitutions in the JAK2 protein, respectively.

Biological Sample Collection and Preparation

The methods and compositions of this invention may be used to detect mutations in the JAK2 gene and/or JAK2 protein using a biological sample obtained from an individual. The nucleic acid (DNA or RNA) may be isolated from the sample according to any methods well known to those of skill in the art. If necessary the sample may be collected or concentrated by centrifugation and the like. The cells of the sample may be subjected to lysis, such as by treatments with enzymes, heat, surfactants, ultrasonication, or a combination thereof. The lysis treatment is performed in order to obtain a sufficient amount of DNA derived from the individual's cells to detect using polymerase chain reaction. Alternatively, mutations in the JAK2 gene may be detected using an acellular bodily fluid according to the methods described in U.S. patent application Ser. No. 11/408,241, hereby incorporated by reference.

Various methods of DNA extraction are suitable for isolating the DNA or RNA. Suitable methods include phenol and chloroform extraction. See Maniatis et al., Molecular Cloning, A Laboratory Manual, 2d, Cold Spring Harbor Laboratory Press, page 16.54 (1989). Numerous commercial kits also yield suitable DNA and RNA including, but not limited to, QIAamp™ mini blood kit, Agencourt Genfind™, Roche Cobast Roche MagNA Pure® or phenol:chloroform extraction using Eppendorf Phase Lock Gels®, and the NucliSens extraction kit (Biomerieux, Marcy l'Etoile, France). In other methods, mRNA may be extracted from patient blood/bone marrow samples using MagNA Pure LC mRNA US kit and Mag NA Pure LC Instrument (Roche Diagnostics Corporation, Roche Applied Science, Indianapolis, Ind.).

Nucleic Acid Extraction and Amplification

Nucleic acid extracted from tissues, cells., plasma or serum can be amplified using nucleic acid amplification techniques well know in the art. Many of these amplification methods can also be used to detect the presence of mutations simply by designing oligonucleotide primers or probes to interact with or hybridize to a particular target sequence in a specific manner. By way of example, but not by way of limitation these techniques can include the polymerase chain reaction (PCR) reverse transcriptase polymerase chain reaction (RT-PCR), nested PCR, ligase chain reaction. See Abravaya, K., et al., Nucleic Acids Research 23:675-682, (1995), branched DNA signal amplification, Urdea, M. S., et al., AIDS 7 (suppl 2):S11-S 14, (1993), amplifiable RNA reporters, Q-beta replication, transcription-based amplification, boomerang DNA amplification, strand displacement activation, cycling probe technology, isothermal nucleic acid sequence based amplification (NASBA). See Kievits, T. et al., J Virological Methods 35:273-286, (1991), Invader Technology, or other sequence replication assays or signal amplification assays.

Reverse Transcription of RNA to cDNA

Some methods employ reverse transcription of RNA to cDNA. As noted, the method of reverse transcription and amplification may be performed by previously published or recommended procedures, which referenced publications are incorporated herein by reference in their entirety. Various reverse transcriptases may be used, including, but not limited to, MMLV RT, RNase H mutants of MMLV RT such as Superscript and Superscript II (Life Technologies, GIBCO BRL, Gaithersburg, Md.), AMV RT, and thermostable reverse transcriptase from Thermus Thermophilus. For example, one method, but not the only method, which may be used to convert RNA extracted from plasma or serum to cDNA is the protocol adapted from the Superscript II Preamplification system (Life Technologies, GIBCO BRL, Gaithersburg, Md.; catalog no. 18089-011), as described by Rashtchian, A., PCR Methods Applic. 4:S83-S91, (1994), adapted as follows.

One (1) to five (5) micrograms of RNA extracted from plasma or serum in 13 μl of DEPC-treated water is added to a clean microcentrifuge tube. Then one microliter of either oligo (dT) (0.5 mg/ml) or random hexamer solution (50 ng/μl) is added and mixed gently. The mixture is then heated to 70 degrees centigrade for 10 minutes and then incubated on ice for one minute. Then, it is centrifuged briefly followed by the addition of 2 μl of 10× synthesis buffer (200 mM Tris-HCl, pH 8.4, 500 mM KCl, 25 mm magnesium chloride, 1 mg/ml of BSA), 1 μl of 10 mM each of dNTP mix, 2 μl of 0.1 M DTT, 1 μl of SuperScript II RT (200 U/μl) (Life Technologies, GIBCO BRL, Gaithersburg, Md.). After gentle mixing, the reaction is collected by brief centrifugation, and incubated at room temperature for 10 minutes. The tube is then transferred to a 42° C. water bath or heat block and incubated for 50 minutes. The reaction is then terminated by incubating the tube at 70° C. for 15 minutes, and then placing it on ice. The reaction is collected by brief centrifugation, and 1 μl of RNase H (2 units) is added followed by incubation at 37° C. for 20 minutes before proceeding to nucleic acid amplification.

Nucleic Acid Amplification

To the cDNA mixture add the following: 8 μl of 10× synthesis buffer (200 mM Tris-HCl, pH 8.4, 500 mM KCl, 25 mM magnesium chloride, 1 mg/ml of BSA), 68 μl sterile double-distilled water, 1 μl amplification primer 1 (10 M), 1 μl amplification primer 2 (10 μM) 1 μl Taq DNA polymerase (2-5 U/μl). Mix gently and overlay the reaction mixture with mineral oil. The mixture is heated to 94° C. for 5 minutes to denature remaining RNA/cDNA hybrids. PCR amplification is then performed in an automated thermal-cycler for 15-50 cycles, at 94° C. for 1 minute, 55° for 30 to 90 seconds, and 72° C. for 2 minutes.

Cycling parameters and magnesium concentration may vary depending upon the specific sequence to be amplified, however, optimization procedures and methods are also well known in the art.

Also, primers may contain appropriate restriction sites, and restriction digestion may be performed on the amplified product to allow further discrimination between mutant and wild-type sequences.

Alternative Methods

Alternative methods of nucleic acid amplification which may be used include variations of RT-PCR, including quantitative RT-PCR, for example as adapted to the method described by Wang, A. M. et al., PNAS USA 86:9717-9721, (1989), or by Karet, F. E., et al. Analytical Biochemistry 220:384-390, (1994).

An alternative method of nucleic acid amplification or mutation detection which may be used is ligase chain reaction (LCR), as described by Wiedmann, et al., PCR Methods Appl. 3:551-564, (1994). In the ligase chain reaction, RNA extracted from plasma or serum is reverse transcribed to cDNA. LCR is a technique to detect single base mutations. A primer is synthesized in two fragments and annealed to the template with possible mutation at the boundary of the two primer fragments. Ligase will ligate the two fragments if they match exactly to the template sequence. Subsequent PCR reactions will amplify only if the primer is ligated. Restriction sites can also be utilized to discriminate between mutant and wild-type sequences.

An alternative method of amplification or mutation detection is allele specific PCR (ASPCR). ASPCR which utilizes matching or mismatching between the template and the 3′ end base of a primer well known in the art. See e.g., U.S. Pat. No. 5,639,611.

Another alternative method of amplification or mutation detection which may be used is branched DNA signal amplification, for example as adapted to the method described by Urdea, M. S., et al., AIDS 7 (suppl 2):S11-S 14, (1993), with modification from the reference as follows: RNA is extracted from plasma or serum and then added directly to microwells. The method for detection of tumor-related or tumor-associated RNA then proceeds as referenced in Urdea, et al, Id., with target probes specific for the tumor-related or tumor-associated RNA or cDNA of interest, and with chemiluminescent light emission proportional to the amount of tumor-associated RNA in the plasma or serum specimen. The specifics of the referenced method arc described further by Urdea, M. S., et al., Nucleic Acids Research Symposium Series 24:197-200, (1991), with this reference incorporated herein in its entirety.

An alternative method of either amplification or mutation detection which may be used is isothermal nucleic acid sequence based amplification (NASBA), for example as adapted to the method described by Kievits, T., et al., J Virological Methods 35:273-286, (1991), or by Vandamme, A. M., et al., J. Virological Methods 52:121-132, (1995).

Alternative methods of either qualitative or quantitative amplification of nucleic acids which may be used, but are not limited to, Q-beta replication, other self-sustained sequence replication assays, transcription-based amplification assays, and amplifiable RNA reporters, boomerang DNA amplification, strand displacement activation, and cycling probe technology.

Another method of mutation detection is nucleic acid sequencing. Sequencing can be performed using any number of methods, kits or systems known in the art. One example is using dye terminator chemistry and an ABI sequencer (Applied Biosystems, Foster City, Calif.). Sequencing also may involve single base determination methods such as single nucleotide primer extension (“SNapShot” sequencing method) or allele or mutation specific PCR.

Exemplary Methods for Detection of JAK2 Nucleic Acid Mutations

Nucleic acid (e.g., total nucleic acid) may be extracted from patient's biological sample using any appropriate method. Next, an RT-PCR reaction may be performed using either the total nucleic acid preparation or the RNA preparation to specifically amplify a portion of the patient RNA. An exemplary one-step RT-PCR system is the Superscript III System (Invitrogen, Carlsbad, Calif.). Other methods and systems for RT-PCR reactions are well known in the art and are commercially available. A primer pair is designed to encompass a region of interest, for example, nucleotides 1920-1923 of SEQ ID NO: 1, to yield a PCR product. By way of example, but not by way of limitation, a primer pair for JAK2 may be 5′-GAC TAC GGT CAA CTG CAT GAA A-3′ (SEQ ID NO: 5), and 5′-CCA TGC CAA CTG TTT AGC AA-3′ (SEQ ID NO: 6). The resulting RT-PCR product is 273 nucleotides long. The RT-PCR product may then be purified, for example by gel purification, and the resulting purified product may be sequenced. Nucleic acid sequencing methods are known in the art; an exemplary sequencing method includes the ABI Prism BIgDye Terminator v3.1 Cycle Sequencing Kit (Applied Biosystems, Foster City, Calif.). The sequencing data may then be analyzed for the presence or absence of one or more mutations in JAK2 nucleic acid. The sequencing data may also be analyzed to determine the proportion of wild-type to mutant nucleic acid present in the sample.

Detection of Mutated JAK2 Proteins

Detection of mutated JAK2 proteins can be accomplished using, for example, antibodies, aptamers, ligands/substrates, other proteins or protein fragments, or other protein-binding agents. Preferably, protein detection agents are specific for the mutated JAK2 protein of the present invention and can therefore discriminate between a mutated protein and the wild-type protein or another variant for. This can generally be accomplished by, for example, selecting or designing detection agents that bind to the region of a protein that differs between the variant and wild-type protein.

One preferred agent for detecting a mutated JAK2 protein is an antibody capable of selectively binding to a variant form of the protein. Antibodies capable of distinguishing between wild-type and mutated JAK2 protein may be created by any suitable method known in the art. The antibodies may be monoclonal or polyclonal antibodies, single chain or double chain, chimeric or humanised antibodies or portions of immunoglobulin molecules containing the portions known in the state of the art to correspond to the antigen binding fragments

Methods for manufacturing polyclonal antibodies are well known in the art. Typically, antibodies are created by administering (e.g., via subcutaneous injection) the mutated JAK2 protein to white New Zealand rabbits. The JAK2 antigen is typically injected at multiple sites and the injections are repeated multiple times (e.g, approximately bi-weekly) to induce an immune response. Desirably, the rabbits are simultaneously administered an adjuvant to enhance anti-JAK2 immunity. The polyclonal antibodies are then purified from a serum sample, for example, by affinity chromatography using the same JAK2 antigen to capture the antibodies.

In vitro methods for detection of the mutated JAK2 proteins also include, for example, enzyme linked immunosorbent assays (ELISAs), radioimmunoassays (RIA), Western blots, immunoprecipitations, immunofluorescence, and protein arrays/chips (e.g., arrays of antibodies or aptamers). For further information regarding immunoassays and related protein detection methods, see Current Protocols in Immunology, John Wiley & Sons, N.Y., and Hage, “Immunoassays”, Anal Chem. Jun. 15, 1999; 71(12):294R-304R. Additional analytic methods of detecting amino acid variants include, but are not limited to, altered dlectrophoretic mobility (e.g., 2-dimensional electrophoresis), altered tryptic peptide digest, altered JAK2 kinase activity in cell-based or cell-free assay, alteration in ligand or antibody-binding pattern, altered isoelectric point, and direct amino acid sequencing.

Diagnostic Tools

The JAK2 nucleic acids of this invention include, for example, nucleic acids that are substantially identical to a portion of the JAK2 nucleotide sequence of SEQ ID NO: 1 and further comprise one or more of the following mutations: G1920A, G1920T, G1920T/C0922T, and T1923C, or complements thereof These nucleic acids may be used as tools to diagnose an individual as having (or as likely to develop) a myeloproliferative disease. Alternatively, the JAK2 mutation status, used alone or in combination with other clinical parameters, also may be used to determine a prognosis for a patient diagnosed as having a myeloproliferative disease. In preferred embodiments, the JAK2 nucleic acids have at least 10, 12, 14, 16, 18, 20, 25, 30, 40, 50, 75, 100, or more nucleotides.

In other preferred embodiments, the JAK2 nucleic acids further comprise a detectable label and are used as a probe (“JAK2 probe”) to detect mutated JAK2 nucleic acids in a patient sample. The JAK2 probe may be detectably labeled by methods known in the art. Useful labels include, for example, fluorescent dyes (e.g., Cy5®, Cy3®, FITC, rhodamine, lanthamide phosphors, Texas red, FAM, JOE, Cal Fluor Red 610®, Quasar 670®), radioisotopes (e.g., ³²P, ³⁵S, ³H, ¹⁴C, ¹²⁵I, ¹³¹I), electron-dense reagents (e.g., gold), enzymes (e.g., horseradish peroxidase, beta-gal actosidase, luciferase, alkaline phosphatase), colorimetric labels (e.g., colloidal gold)., magnetic labels (e.g., Dynabeads™), biotin, dioxigenin, or haptens and proteins for which antisera or monoclonal antibodies are available. Other labels include ligands or oligonucleotides capable of forming a complex with the corresponding receptor or oligonucleotide complement, respectively. The label can be directly incorporated into the nucleic acid to be detected, or it can be attached to a probe (e.g., an oligonucleotide) or antibody that hybridizes or binds to the nucleic acid to be detected.

In other preferred embodiments, the JAK2 probes are TaqMan® probes, molecular beacons, and Scorpions (e.g., Scorpion™ probes). These types of probes are based on the principle of fluorescence quenching and involve a donor fluorophore and a quenching moiety. The term “fluorophore” as used herein refers to a molecule that absorbs light at a particular wavelength (excitation frequency) and subsequently emits light of a longer wavelength (emission frequency). The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety.

The term “quencher moiety” as used herein means a molecule that, in close proximity to a donor fluorophore, takes up emission energy generated by the donor and either dissipates the energy as heat or emits light of a longer wavelength than the emission wavelength of the donor. In the latter case, the quencher is considered to be an acceptor fluorophore. The quenching moiety can act via proximal (i.e., collisional) quenching or by Forster or fluorescence resonance energy transfer (“FRET”). Quenching by FRET is generally used in TaqMan® probes while proximal quenching is used in molecular beacon and Scorpion™ type probes. Suitable quenchers are selected based on the fluorescence spectrum of the particular fluorophore. Useful quenchers include, for example, the Black Hole™ quenchers BHQ-1, BHQ-2, and BHQ-3 (Bioscarch Technologies, Inc.), and the ATTO-series of quenchers (ATTO 540Q, ATTO 580Q, and ATTO 612Q; Atto-Tec GmbH).

With Scorpion primers, sequence-specific priming and PCR product detection is achieved using a single molecule. The Scorpion primer maintains a stem-loop configuration in the unhybridized state. The fluorophore is attached to the 5′ end and is quenched by a moiety coupled to the 3′ end, although in suitable embodiments, this arrangement may be switched The 3′ portion of the stem also contains sequence that is complementary to the extension product of the primer. This sequence is linked to the 5′ end of a specific primer via a non-amplifiable monomer. After extension of the primer moiety, the specific probe sequence is able to bind to its complement within the extended amplicon thus opening up the hairpin loop. This prevents the fluorescence from being quenched and a signal is observed. A specific target is amplified by the reverse primer and the primer portion of the Scorpion primer, resulting in an extension product. A fluorescent signal is generated due to the separation of the fluorophore from the quencher resulting from the binding of the probe element (e.g., the JAK2 probe) of the Scorpion primer to the extension product.

TaqMan® probes (Heid, et al., Genome Res 6: 986-994, 1996) use the fluorogcnic 5′ exonuclease activity of Taq polymerase to measure the amount of target sequences in cDNA samples. TaqMan® probes are oligonucleotides that contain a donor fluorophore usually at or near the 5′ base, and a quenching moiety typically at or near the 3′ base. The quencher moiety may be a dye such as TAMRA or may be a non-fluorescent molecule such as 4-(4 -dimethylaminophenylazo) benzoic acid (DABCYL). See Tyagi, et al., 16 Nature Biotechnology 49-53 (1998). When irradiated, the excited fluorescent donor transfers energy to the nearby quenching moiety by FRET rather than fluorescing. Thus, the close proximity of the donor and quencher prevents emission of donor fluorescence while the probe is intact.

TaqMan® probes are designed to anneal to an internal region of a PCR product. When the polymerase (e.g., reverse transcriptase) replicates a template on which a TaqMan® probe is bound, its 5′ exonuclease activity cleaves the probe. This ends the activity of the quencher (no FRET) and the donor fluorophore starts to emit fluorescence which increases in each cycle proportional to the rate of probe cleavage. Accumulation of PCR product is detected by monitoring the increase in fluorescence of the reporter dye (note that primers are not labeled). If the quencher is an acceptor fluorophore, then accumulation of PCR product can be detected by monitoring the decrease in fluorescence of the acceptor fluorophore.

Kits for Detecting JAK2 Mutations

The invention also provides kits for detecting JAK2 mutations. The kits will contain at least a primer pair capable of amplifying a target JAK2 nucleic acid sequence that encompasses nucleotides 1920-1923 of SEQ ID NO: 1 and a means for detecting a JAK2 mutation in the target. Preferably, the amplification using the primer pair of the kit results in a reaction product having at least 20, 40, 60, 80, 100, 125, 150, 200, 300, 500, or more nucleotides. Suitable primer pairs include, for example, primers having the sequence of SEQ ID NOs: 5 and 6. Suitable means for detecting a JAK2 mutation in the reaction product the use of a detectably labeled JAK2 probe, such as those described herein.

Diagnosis and Prognosis

The presence of the JAK2 mutations, including, for example, the V617F, V617I and C618R mutations alone, in combination with each other, or in combination with other JAK2 mutations can be as an indicator of disease. Additionally, the zygosity status of these mutations is prognostic of disease progression and patient longevity for some patient populations. The ratio of the mutant to wild-type nucleic acid in a patient sample may be used to monitor facts such as disease progression and treatment efficacy.

The zygosity status and the ratio of wild-type to mutant nucleic acid in a sample may be determined by methods known in the art including sequence-specific, quantitative detection methods. Other methods may involve determining the area under the curves of the sequencing peaks from standard sequencing electropherograms, such as those created using ABI Sequencing Systems, (Applied Biosystems, Foster City Calif.). For example, the presence of only a single peak such as a “G” on an electropherogram in a position representative of a particular nucleotide is an indication that the nucleic acids in the sample contain only one nucleotide at that position, the “G.” The sample may then be categorized as homozygous because only one allele is detected. The presence of two peaks, for example, a “G” peak and a “T” peak in the same position on the electropherogram indicates that the sample contains two species of nucleic acids; one species carries the “G” at the nucleotide position in question, the other carries the “T” at the nucleotide position in question. The sample may then be categorized as heterozygous because more than one allele is detected.

The sizes of the two peaks maybe determined (e.g, by determining the area under each curve), and a ratio of the two different nucleic acid species may be calculated. A ratio of wild-type to mutant nucleic acid may be used to monitor disease progression, determine treatment or to make a diagnosis. For example, the number of cancerous cells carrying one or more of the mutations identified herein may change during the course of an myeloproliferative disease. If a base line ratio is established early in the disease, a later determined higher ratio of mutant nucleic acid relative to wild-type nucleic acid may be an indication that the disease is becoming worse or a treatment is ineffective; the number of cells carrying the mutation may be increasing in the patient. A lower ratio of mutant relative to wild-type nucleic acid may be an indication that a treatment is working or that the disease is not progressing; the number of cells carrying the mutation may be decreasing in the patient.

### EXAMPLES

### Example 1

### Detection of the JAK2 Mutations

Whole blood samples of 634 individuals of unknown MPD status were tested for JAK2 mutations in the region surrounding the codon which encodes amino acid 617 of SEQ ID NO: 3. Also tested were 130 plasma samples from patients with confirmed MPD.

Total RNA was extracted from the mixtures using the NucliSense Extraction Kit (bioMericux Inc., Durham, N.C.) as recommended by the manufacturer. A PCR primer pair was designed to amplify across the region of the JAK2 gene coding for amino acid 671. The primer sequences used for PCR and sequencing were as follows: JAK2-F (5′-GAC TAC GGT CAA CTG CAT GAA A-3′) SEQ ID NO: 5 (corresponding to nucleotides 1776-1797 of SEQ ID NO: 1), and JAK2-R (5′-CCA TGC CAA CTG TTT AGC AA-3′) SEQ ID NO: 6 (corresponding to nucleotides 2029-2048 of SEQ ID NO: 1). One-step RT-PCR was performed in a 25 μL reaction volume using SuperScript III one-step RT-PCR system with Platinum Taq (Invitrogen, Carlsbad, Calif.). Concentrations used for RT-PCR were: 1× reaction buffer, 400 nM each of the forward and reverse JAK2 primers, 1 unit of SupersScript III and 5 μL of the RNA template. The thermocycler conditions were: 30 minutes at 55° C. for reverse transcription, followed by 2 minutes at 94° C. and 40 cycles of 94° C. for 15 seconds, 60° C. for 30 seconds, 68° C. for 1 minute, with a final step of 68° C. for 7 minutes.

The amplification product was filtration purified using a Multiscreen PCR plate (Millipore, Billerica, Mass.) and then sequenced in both forward and reverse directions using the ABI Prism Big Dye Terminator V3.1 Cycle Sequencing Kit and the ABI PRISM 3100 Genetic Analyzer (Applied Biosystems, Foster City Calif.) using the JAK2 sequence in GenBank accession number NM004972 as a reference.

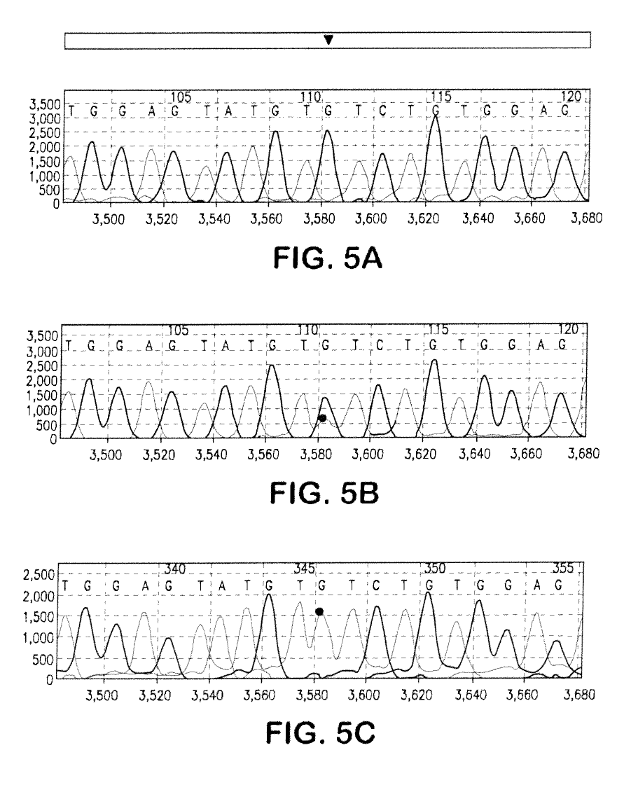

Most commonly detected was the V617F amino acid substitution in the JAK2 protein resulting from the G1920T mutation. FIG. 5 shows representative electropherogram tracings from cell- and plasma-based JAK2 mutational analysis. FIG. 5A shows a tracing from a normal (wild-type) individual. FIG. 5B and 5C show the tracings from a blood cell and plasma sample of an individual that that harbors the G1920T mutation. FIG. 5B indicates that the individual is heterozygous for the G1920T mutation. The dot indicates the peak associated with the thiamine base. However, FIG. 5C obtained from a plasma sample of the same individual indicates that the individual is homozygous or hemizygous for the mutation. The spurious result from the blood cell sample is likely the result of a high level of normal blood cells which dilute/contaminate the leukemic cell nucleic acids. Plasma is, therefore, enriched in tumor-specific nucleic acid and provides a more reliable assay substrate for detecting MPD.

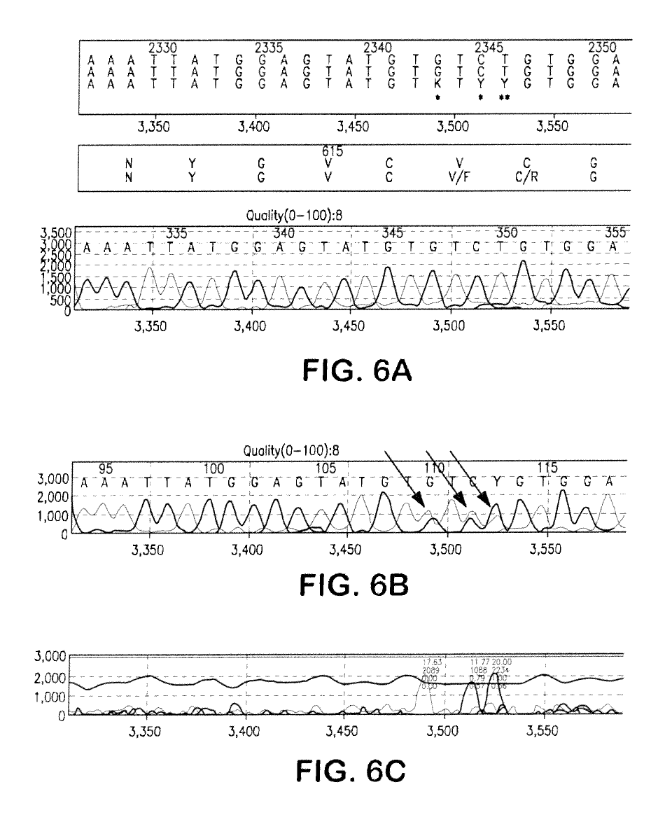

Two novel JAK2 point mutations were discovered in a single patient. As shown in FIG. 6, the patient harbored both a variant nucleic acid mutation resulting in the V617F amino acid substitution, and a second nucleic acid mutation resulting in the C618R amino acid substitution. FIG. 6A shows an electropherogram tracing from a patient having normal (wild-type) JAK2. FIG. 6B shows a double point mutation in the codon encoding amino acid 617. Specifically, the wild-type GTC codon (valine) contains the G1920T/C1922T double point mutation (grey arrows), resulting in a TTT codon (arginine). The individual was also discovered to have the T1923C point mutation (black arrow) resulting in a codon change from TGT (cysteine) to CGT (arginine).

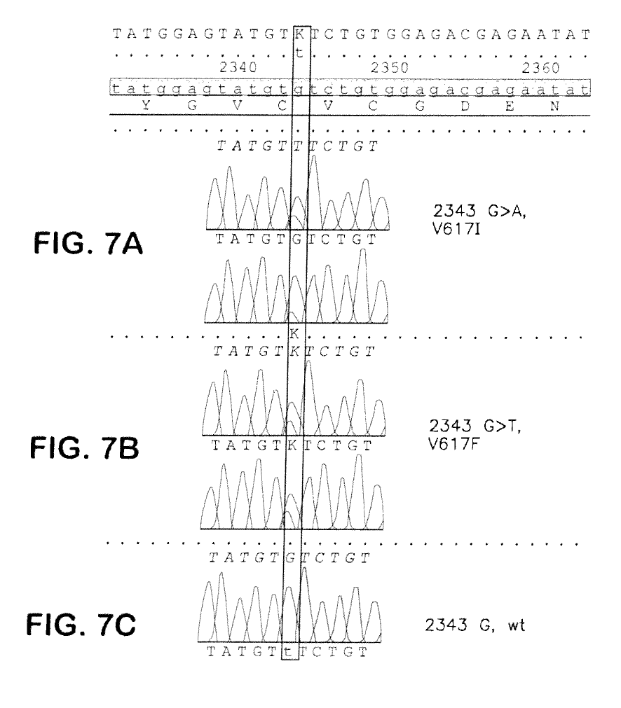

Another novel JAK2 point mutation was discovered in a different patient. As shown in FIG. 7, this patient has a G1920A point mutation, resulting in the V617I amino acid substitution in the JAK2 protein.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

What is claimed is:

1. A method for diagnosing a neoplastic disease comprising determining the presence or absence of one or more mutations in the JAK2 nucleic acid of a patient, said mutation selected from the group consisting of G1920A, T1923C, and G1920T/C1922T.

2. The method of claim 1, wherein said mutation is G1920A.

3. The method of claim 1, wherein said mutation is G1920T/C1922T.

4. The method of claim 1, wherein said mutation is T1923C.

5. The method of claim 4, wherein said JAK2 nucleic acid further comprises the G1920T mutation.

6. The method of claim 4, wherein said JAK2 nucleic acid further comprises the G1920T/C1922T mutation.

7. The method of claim 4, wherein said JAK2 nucleic acid further comprises the T1920A mutation.

8. The method of claim 1, wherein said neoplastic disease is a myeloproliferative disease.

9. The method of claim 8, wherein myeloproliferative disease is selected from the group consisting of polycythemia vera, essential thrombocythemia, idiopathic myleofibrosis, and unclassified myeloproliferative disease.

10. The method of claim 1, wherein said mutation affects JAK2 kinase activity.

11. A method for determining a prognosis of an individual diagnosed with a neoplastic disease comprising determining the presence or absence of one or more mutations in the JAK2 nucleic acid of a patient, said mutation selected from the group consisting of G1920A, T1923C, and G1920T/C1922T, and using the mutation status to predict the clinical outcome for the individual.

12. The method of claim 11, wherein said mutation is G1920A.

13. The method of claim 11, wherein said mutation is G1920T/C1922T.

14. The method of claim 11, wherein said mutation is T1923C.

15. The method of claim 14, wherein said JAK2 nucleic acid further comprises the G1920T mutation.

16. The method of claim 14, wherein said JAK2 nucleic acid further comprises the G1920T/C1922T mutation.

17. The method of claim 14, wherein said JAK2 nucleic acid further comprises the T1920A mutation.

18. The method of claim 11, wherein said neoplastic disease is a myeloproliferative disease.

19. The method of claim 18, wherein myeloproliferative disease is selected from the group consisting of polycythemia vera, essential thrombocythemia, idiopathic myleofibrosis, and unclassified myeloproliferative disease.

20. The method of claim 11, wherein said mutation affects JAK2 kinase activity.

21. The method of claim 11, wherein the mutation status is combined with at least one other clinical parameter to determine the clinical outcome for the individual.

22. The method of claim 21, wherein at least one other clinical parameter is selected from the group consisting of age and percent blast cell count.

23. A method for diagnosing a neoplastic disease comprising determining the presence or absence of a mutation in the JAK2 protein of a patient, said mutation selected from the group consisting of V617A and C618R.

24. The method of claim 23, wherein said mutation is V617A.

25. The method of claim 23, wherein said mutation is C618R.

26. The method of claim 25, wherein said JAK2 protein further comprises a V617F mutation.

27. The method of claim 23, wherein said neoplastic disease is a myeloproliferative disease.

28. The method of claim 27, wherein myeloproliferative disease is selected from the group consisting of polycythemia vera, essential thrombocythemia, idiopathic myleofibrosis, and unclassified myeloproliferative disease.

29. The method of claim 23, wherein said mutation affects JAK2 kinase activity.

30. An isolated nucleic acid comprising at least 14 nucleotides of SEQ ID NO: 1, wherein said nucleic acid comprises a mutation selected from the group consisting of G1920A, T1923C, and G1920T/C1922T, or a complement thereof.

31. The nucleic acid of claim 30, wherein said mutation is G1920A.

32. The nucleic acid of claim 30, wherein said mutation is G1920T/C1922T.

33. The nucleic acid of claim 30, wherein said mutation is T1923C.

34. The nucleic acid of claim 33, wherein said nucleic acid further comprises the G1920T mutation.

35. The nucleic acid of claim 33, wherein said nucleic acid further comprises the G1920T/C1922T mutation.

36. The nucleic acid of claim 33, wherein said nucleic acid further comprises the T1920A mutation.

37. The nucleic acid of claim 30, wherein said nucleic acid comprises at least 50 nucleotides.

38. The nucleic acid of claim 30, wherein said nucleic acid further comprises a detectable label.

39. An isolated polypeptide comprising at least 10 contiguous amino acids of SEQ ID NO: 3 wherein said polypeptide comprises a mutation selected from the group consisting of V617I and C618R.

40. The polypeptide of claim 39, wherein said mutation is V617I.

41. The polypeptide of claim 39, wherein said mutation is C618R.

42. The polypeptide of claim 41, wherein said polypeptide further comprises the V617I mutation.

43. The polypeptide of claim 39, wherein said polypeptide comprises at least 50 amino acids.


SEQUENCE LISTING (18 sequences)

SEQ ID NO: 1 - DNA, 3652 bp, Homo sapiens
tttctcttctgcagaaaaagaggctcttcctcctcctcccgcgacggcaaatgttctgaaaaagactctgcatgggaatggcctgccttacgatgacagaaatggagggaacatccacctcttctatatatcagaatggtgatatttctggaaatgccaattctatgaagcaaatagatccagttcttcaggtgtatctttaccattcccttgggaaatctgaggcagattatctgacctttccatctggggagtatgttgcagaagaaatctgtattgctgcttctaaagcttgtggtatcacacctgtgtatcataatatgtttgctttaatgagtgaaacagaaaggatctggtatccacccaaccatgtcttccatatagatgagtcaaccaggcataatgtactctacagaataagattttactttcctcgttggtattgcagtggcagcaacagagcctatcggcatggaatatctcgaggtgctgaagctcctcttcttgatgactttgtcatgtcttacctctttgctcagtggcggcatgattttgtgcacggatggataaaagtacctgtgactcatgaaacacaggaagaatgtcttgggatggcagtgttagatatgatgagaatagccaaagaaaacgatcaaaccccactggccatctataactctatcagctacaagacattcttaccaaaatgtattcgagcaaagatccaagactatcatattttgacaaggaagcgaataaggtacagatttcgcagatttattcagcaattcagccaatgcaaagccactgccagaaacttgaaacttaagtatcttataaatctggaaactctgcagtctgccttctacacagagaaatttgaagtaaaagaacctggaagtggtccttcaggtgaggagatttttgcaaccattataataactggaaacggtggaattcagtggtcaagagggaaacataaagaaagtgagacactgacagaacaggatttacagttatattgcgattttcctaatattattgatgtcagtattaagcaagcaaaccaagagggttcaaatgaaagccgagttgtaactatccataagcaagatggtaaaaatctggaaattgaacttagctcattaagggaagctttgtctttcgtgtcattaattgatggatattatagattaactgcagatgcacatcattacctctgtaaagaagtagcacctccagccgtgcttgaaaatatacaaagcaactgtcatggcccaatttcgatggattttgccattagtaaactgaagaaagcaggtaatcagactggactgtatgtacttcgatgcagtcctaaggactttaataaatattttttgacttttgctgtcgagcgagaaaatgtcattgaatataaacactgtttgattacaaaaaatgagaatgaagagtacaacctcagtgggacaaagaagaacttcagcagtcttaaagatcttttgaattgttaccagatggaaactgttcgctcagacaatataattttccagtttactaaatgctgtcccccaaagccaaaagataaatcaaaccttctagtcttcagaacgaatggtgtttctgatgtaccaacctcaccaacattacagaggcctactcatatgaaccaaatggtgtttcacaaaatcagaaatgaagatttgatatttaatgaaagccttggccaaggcacttttacaaagatttttaaaggcgtacgaagagaagtaggagactacggtcaactgcatgaaacagaagttcttttaaaagttctggataaagcacacagaaactattcagagtctttctttgaagcagcaagtatgatgagcaagctttctcacaagcatttggttttaaattatggagtatgtgtctgtggagacgagaatattctggttcaggagtttgtaaaatttggatcactagatacatatctgaaaaagaataaaaattgtataaatatattatggaaacttgaagttgctaaacagttggcatgggccatgcattttctagaagaaaacacccttattcatgggaatgtatgtgccaaaaatattctgcttatcagagaagaagacaggaagacaggaaatcctcctttcatcaaacttagtgatcctggcattagtattacagttttgccaaaggacattcttcaggagagaataccatgggtaccacctgaatgcattgaaaatcctaaaaatttaaatttggcaacagacaaatggagttttggtaccactttgtgggaaatctgcagtggaggagataaacctctaagtgctctggattctcaaagaaagctacaattttatgaagataggcatcagcttcctgcaccaaagtgggcagaattagcaaaccttataaataattgtatggattatgaaccagatttcaggccttctttcagagccatcatacgagatcttaacagtttgtttactccagattatgaactattaacagaaaatgacatgttaccaaatatgaggataggtgccctggggttttctggtgcctttgaagaccgggatcctacacagtttgaagagagacatttgaaatttctacagcaacttggcaagggtaattttgggagtgtggagatgtgccggtatgaccctctacaggacaacactggggaggtggtcgctgtaaaaaagcttcagcatagtactgaagagcacctaagagactttgaaagggaaattgaaatcctgaaatccctacagcatgacaacattgtaaagtacaagggagtgtgctacagtgctggtcggcgtaatctaaaattaattatggaatatttaccatatggaagtttacgagactatcttcaaaaacataaagaacggatagatcacataaaacttctgcagtacacatctcagatatgcaagggtatggagtatcttggtacaaaaaggtatatccacagggatctggcaacgagaaatatattggtggagaacgagaacagagttaaaattggagattttgggttaaccaaagtcttgccacaagacaaagaatactataaagtaaaagaacctggtgaaagtcccatattctggtatgctccagaatcactgacagagagcaagttttctgtggcctcagatgtttggagctttggagtggttctgtatgaacttttcacatacattgagaagagtaaaagtccaccagcggaatttatgcgtatgattggcaatgacaaacaaggacagatgatcgtgttccatttgatagaacttttgaagaataatggaagattaccaagaccagatggatgcccagatgagatctatatgatcatgacagaatgctggaacaataatgtaaatcaacgcccctcctttagggatctagctcttcgagtggatcaaataagggataacatggctggatgaaagaaatgaccttcattctgagaccaaagtagatttacagaacaaagttttatatttcacattgctgtggactattattacatatatcattattatataaatcatgatgctagccagcaaagatgtgaaaatatctgctcaaaactttcaaagtttagtaagtttttcttcatgaggccacc

SEQ ID NO: 2 - DNA, 879 bp, Homo sapiens
cacaaaatcagaaatgaagatttgatatttaatgaaagccttggccaaggcacttttacaaagatttttaaaggcgtacgaagagaagtaggagactacggtcaactgcatgaaacagaagttcttttaaaagttctggataaagcacacagaaactattcagagtctttctttgaagcagcaagtatgatgagcaagctttctcacaagcatttggttttaaattatggagtatgtgtctgtggagacgagaatattctggttcaggagtttgtaaaatttggatcactagatacatatctgaaaaagaataaaaattgtataaatatattatggaaacttgaagttgctaaacagttggcatgggccatgcattttctagaagaaaacacccttattcatgggaatgtatgtgccaaaaatattctgcttatcagagaagaagacaggaagacaggaaatcctcctttcatcaaacttagtgatcctggcattagtattacagttttgccaaaggacattcttcaggagagaataccatgggtaccacctgaatgcattgaaaatcctaaaaatttaaatttggcaacagacaaatggagttttggtaccactttgtgggaaatctgcagtggaggagataaacctctaagtgctctggattctcaaagaaagctacaattttatgaagataggcatcagcttcctgcaccaaagtgggcagaattagcaaaccttataaataattgtatggattatgaaccagatttcaggccttctttcagagccatcatacgagatcttaacagtttgtttactccagattatgaactattaacagaaaatgacatgttaccaaatatgaggataggtgccctg

SEQ ID NO: 3 - protein, 1132 aa, Homo sapiens
MGMACLTMTEMEGTSTSSIYQNGDISGNANSMKQIDPVLQVYLYHSLGKSEADYLTFPSGEYVAEEICIAASKACGITPVYHNMFALMSETERIWYPPNHVFHIDESTRHNVLYRIRFYFPRWYCSGSNRAYRHGISRGAEAPLLDDFVMSYLFAQWRHDFVHGWIKVPVTHETQEECLGMAVLDMMRIAKENDQTPLAIYNSISYKTFLPKCIRAKIQDYHILTRKRIRYRFRRFIQQFSQCKATARNLKLKYLINLETLQSAFYTEKFEVKEPGSGPSGEEIFATIIITGNGGIQWSRGKHKESETLTEQDLQLYCDFPNIIDVSIKQANQEGSNESRVVTIHKQDGKNLEIELSSLREALSFVSLIDGYYRLTADAHHYLCKEVAPPAVLENIQSNCHGPISMDFAISKLKKAGNQTGLYVLRCSPKDFNKYFLTFAVERENVIEYKHCLITKNENEEYNLSGTKKNFSSLKDLLNCYQMETVRSDNIIFQFTKCCPPKPKDKSNLLVFRTNGVSDVPTSPTLQRPTHMNQMVFHKIRNEDLIFNESLGQGTFTKIFKGVRREVGDYGQLHETEVLLKVLDKAHRNYSESFFEAASMMSKLSHKHLVLNYGVCVCGDENILVQEFVKFGSLDTYLKKNKNCINILWKLEVAKQLAWAMHFLEENTLIHGNVCAKNILLIREEDRKTGNPPFIKLSDPGISITVLPKDILQERIPWVPPECIENPKNLNLATDKWSFGTTLWEICSGGDKPLSALDSQRKLQFYEDRHQLPAPKWAELANLINNCMDYEPDFRPSFRAIIRDLNSLFTPDYELLTENDMLPNMRIGALGFSGAFEDRDPTQFEERHLKFLQQLGKGNFGSVEMCRYDPLQDNTGEVVAVKKLQHSTEEHLRDFEREIEILKSLQHDNIVKYKGVCYSAGRRNLKLIMEYLPYGSLRDYLQKHKERIDHIKLLQYTSQICKGMEYLGTKRYIHRDLATRNILVENENRVKIGDFGLTKVLPQDKEYYKVKEPGESPIFWYAPESLTESKFSVASDVWSFGVVLYELFTYIEKSKSPPAEFMRMIGNDKQGQMIVFHLIELLKNNGRLPRPDGCPDEIYMIMTECWNNNVNQRPSFRDLALRVDQIRDNMAG

SEQ ID NO: 4 - protein, 290 aa, Homo sapiens
RNEDLIFNESLGQGTFTKIFKGVRREVGDYGQLHETEVLLKVLDKAHRNYSESFFEAASMMSKLSHKHLVLNYGVCVCGDENILVQEFVKFGSLDTYLKKNKNCINILWKLEVAKQLAWAMHFLEENTLIHGNVCAKNILLIREEDRKTGNPPFIKLSDPGISITVLPKDILQERIPWVPPECIENPKNLNLATDKWSFGTTLWEICSGGDKPLSALDSQRKLQFYEDRHQLPAPKWAELANLINNCMDYEPDFRPSFRAIIRDLNSLFTPDYELLTENDMLPNMRIGAL

SEQ ID NO: 5 - DNA, 22 bp, Artificial Sequence
  Description of Artificial Sequence Synthetic primer
gactacggtcaactgcatgaaa

SEQ ID NO: 6 - DNA, 20 bp, Artificial Sequence
  Description of Artificial Sequence Synthetic primer
ccatgccaactgtttagcaa

SEQ ID NO: 7 - DNA, 20 bp, Homo sapiens
tggagtatgtgtctgtggag

SEQ ID NO: 8 - DNA, 25 bp, Homo sapiens
aaattatggagtatgtgtctgtgga

SEQ ID NO: 9 - DNA, 25 bp, Homo sapiens
aaattatggagtatgtktyygtgga

SEQ ID NO: 10 - protein, 8 aa, Homo sapiens
NYGVCVCG

SEQ ID NO: 11 - protein, 8 aa, Homo sapiens
  MOD_RES (6)..(6): Val or Phe
NYGVCXXG

SEQ ID NO: 12 - DNA, 25 bp, Homo sapiens
aaattatggagtatgtgtcygtgga

SEQ ID NO: 13 - DNA, 32 bp, Homo sapiens
tatggagtatgtktctgtggagacgagaatat

SEQ ID NO: 14 - DNA, 32 bp, Homo sapiens
tatggagtatgtttctgtggagacgagaatat

SEQ ID NO: 15 - DNA, 32 bp, Homo sapiens
tatggagtatgtgtctgtggagacgagaatat

SEQ ID NO: 16 - protein, 10 aa, Homo sapiens
YGVCVCGDEN

SEQ ID NO: 17 - DNA, 11 bp, Homo sapiens
tatgtgtctgt

SEQ ID NO: 18 - DNA, 11 bp, Homo sapiens
tatgtktctgt